(19) United States
(12) Patent Application Publication
(10) Pub. No.: US 2007/0066879 A1
(43) Pub. Date: Mar. 22, 2007

(54) BODY LUMEN SHAPING DEVICE WITH CARDIAC LEADS

(76) Inventors: Mark L. Mathis, Kirkland, WA (US); Gregory D. Nieminen, Bothell, WA (US); David G. Reuter, Bothell, WA (US)

Correspondence Address: WILSON SONSINI GOODRICH & ROSATI, 650 PAGE MILL ROAD, PALO ALTO, CA, 94304-1050, US

(21) Appl. No.: 11/550,354

(22) Filed: Oct. 17, 2006

Related U.S. Application Data
(62) Division of application No. 11/132,786, filed on May 18, 2005.
Application No. 11/132,786 is a continuation-in-part of application No. 10/066,426, filed on Jan. 30, 2002, now Pat. No. 6,976,995.

(51) Int. Cl. A61B 5/042; A61F 2/24; A61N 1/05

(52) U.S. Cl. 600/375; 623/2.36; 607/130

(57) ABSTRACT

A shaping device and a cardiac lead, both adapted to be disposed in a coronary sinus of a patient's heart, are provided. In one method, a patient is treated by deploying in the patient's coronary sinus a shaping device and a cardiac lead and using the shaping device to modify mitral valve geometry.

### CROSS-REFERENCE

This application is a divisional application of U.S. patent application Ser. No. 11/132,786, “Body Lumen Shaping Device With Cardiac Leads”, filed on May 18, 2005, which is a continuation-in-part of U.S. patent application Ser. No. 10/066,426, “Fixed Length Anchor and Pull Mitral Valve Device and Method”, filed Jan. 30, 2002, now U.S. Pat. No. 6,976,995, which is incorporated herein by reference in its entirety and to which application we claim priority under 35 USC § 120.

### FIELD OF THE INVENTION

The present invention generally relates to devices and methods for treating a mitral valve and delivering and/or maintaining one or more electrophysiology (EP) devices, such as a cardiac lead, in a coronary sinus.

### BACKGROUND OF THE INVENTION

The mitral valve is located in the left side of the heart, between the left upper chamber (atrium) and left lower chamber (ventricle). The valve comprises two flaps, called leaflets, which normally close each time the left ventricle contracts in order to pump blood out of the heart. When the mitral valve doesn't close properly, blood from the ventricle is forced back up (i.e. regurgitated) into the left atrium instead of flowing out to the rest of the body. This condition is known as mitral valve, or mitral, regurgitation. The added workload on a patient's heart, and the increased blood pressure in the lungs caused by this heart condition, poses serious health risks if this condition is left untreated.

Failure of the mitral valve leaflets to open and close properly can be caused by damage to the leaflets. For example, rheumatic fever and other infections can damage valve leaflets and cause scarring. These scars can deform the leaflets, preventing the valve from opening and closing properly. In addition, if one or more of the cordlike structures attaching the leaflets to the heart muscles break, the valve may also leak. Heart attacks, diseases of the heart muscle, or other heart valve abnormalities, which can cause enlargement of the entire heart, can stretch the mitral valve annulus and the muscular valve cusp attachments, pulling the valve leaflets apart so that the leaflets no longer meet.

Treatment for mitral valve regurgitation can vary depending on severity of the condition. Mitral valve regurgitation associated with normal heart sizes and without symptoms will be left untreated. This is because conventional treatments for mitral valve regurgitation involve invasive open heart surgery. Moderate to severe mitral valve regurgitation requires treatment, either mitral valve repair or replacement. However, an attendant drawback of surgical valve replacement, or repair, is the high morbidity, cost and trauma associated with these treatments. Surgical valve repair commonly involves narrowing the mitral valve ring or annulus and tailoring the valve leaflets by stitching a plastic support ring around the valve to bring the leaflets closer together. For more damaged leaflets, replacement of the entire mitral valve with a prosthetic valve may be necessary.

Recently, a non-invasive therapy for the treatment of mitral valve regurgitation has been proposed. This treatment is based on the proximity of the coronary sinus to the mitral valve annulus. The coronary sinus at least partially encircles the annulus and extends into the venous system, including the great cardiac vein. As used herein, “coronary sinus” refers to not only the coronary sinus itself, but also to the venous system associated with the coronary sinus including the great cardiac vein. The therapy contemplates the use of an implantable device introduced into the coronary sinus to reshape and advantageously effect the geometry of the annulus and the leaflets. Such devices include those shown and described in U.S. Publication No. 2004/0220654 (Attorney Docket No. 29912-715.201), filed May 2, 2003, and which is incorporated by reference in its entirety. Methods of implantation of such devices include those described in U.S. Publication No. US 2004/0158321 (Atty Docket No. 29912.701.201), filed Feb. 2, 2003 and U.S. application Ser. No. 10/946,332 (Atty Docket No. 29912-710.501), filed Sep. 20, 2004, the entire contents of which are hereby incorporated by reference.

As will be readily appreciated by those skilled in the art, other therapeutic and diagnostic cardiac devices, in addition to those adapted for the treatment of mitral valve regurgitation, are introduced through or deployed in the coronary sinus. For example, various electrophysiology (EP) systems having cardiac leads are often delivered through, or implanted in, the coronary sinus. However, accessing and implanting leads of EP devices to the coronary sinus can be difficult, as these devices are not particularly designed for good pushability. Also, these leads can be difficult to maintain in position within the coronary sinus as movement of the heart during heartbeats tends to cause dislodgement of these components. Moreover, devices and system that are adapted to allow multiple diagnostic and/or therapeutic procedures to be performed simultaneously (such as mitral regurgitation treatment along with cardiac pacing) would provide several advantages.

Therefore, devices and methods that can provide efficient and easy delivery and maintenance of EP device components (or leads) in a coronary sinus and/or allow the performance of multiple therapies simultaneously are needed. The present invention meets these as well as other needs.

### SUMMARY OF THE INVENTION

Generally, the invention relates to the treatment of mitral valve regurgitation and electrophysiology (EP), and in particular to methods, device and systems for deploying a shaping device and cardiac lead inside a patient's heart. As further described herein, the shaping devices of the present invention is adapted to modify or effect mitral valve geometry upon deployment thereby reducing mitral valve regurgitation, while the cardiac leads of the present invention are components of an electrophysiology (EP) system configured to provide sensing, pacing and/or defribrillation of the patient for a variety of therapeutic and/or diagnostic purposes. In a preferred embodiment, the shaping device and/or cardiac leads are deployable within, or passed through, a patient's coronary sinus, although use of the various devices and techniques described herein may be used to treat other valves, body lumens, etc.

In one embodiment, a method for treating a patient's heart comprises deploying in the patient's coronary sinus a shaping device and a cardiac lead and using the shaping device to modify mitral valve geometry. In some examples, the deploying step may comprise deploying the shaping device in the coronary sinus before deploying the cardiac lead in the coronary sinus or vice versa. In yet other examples, the deploying step may comprise passing the cardiac lead through a portion of the shaping device. As is further described herein the shaping device and lead may be deployed simultaneously.

In deploying the shaping device, the cardiac lead can be disposed between a portion of the shaping device and a coronary sinus wall, preferably the sinus wall proximate the left atrium. In yet another example, the step of deploying in the patient's coronary sinus a shaping device may comprise deploying the shaping device and cardiac lead such that a portion of the shaping device surrounds a portion of the cardiac lead. In another example, the step of deploying in the patient's coronary sinus a shaping device may comprise deploying the shaping device and cardiac lead such that the shaping device is adjacent to the cardiac lead. In yet other examples, the step of deploying in the patient's coronary sinus a shaping device may comprise deploying the shaping device and cardiac lead such that the shaping device contacts the cardiac lead.

In yet another of the invention, the cardiac lead may be a cardiac resynchronization therapy lead, a IPG, a ICD, a PCD or pacemaker lead. In yet another embodiment of the invention, the shaping device may further comprise a retention member adapted to hold the cardiac lead within the coronary sinus.

In yet another embodiment of the present invention, a method for treating a patient's heart comprises: deploying in the patient's coronary sinus a shaping device having one more electrodes and which is adapted to couple to a conventional electrophysiology (EP) system; and using the shaping device to modify mitral valve geometry. In some examples, the EP system may be used to defibrillate a right and left ventricle.

In yet another embodiment of the invention, a device for treating a condition of the heart and which is configured to be deployed in a coronary sinus, said device comprising: expandable first and second anchors interconnected by a connecting member disposed between the first and second anchors; and a retention member adapted for holding a cardiac lead inside a coronary sinus. In some examples, the retention member may be a loop, hook, grasper or the like.

In yet another embodiment, a device for treating a condition of a patient's heart and which is configured to be deployed in a coronary sinus, said device comprising: a distal anchor; a proximal anchor; a connecting member disposed between the distal and proximal anchors; one or more electrodes; and a lead wire which operationally couples the one or more electrodes to an EP system. In some examples, the one or more electrodes may be located on the distal anchor, the proximal anchor, on both anchors, on the connector member, or any other combination thereof.

yet another embodiment of the present invention comprises a device that effects mitral valve annulus geometry of a heart, comprising: a first anchor configured to be positioned within and anchored to the coronary sinus of the heart adjacent the mitral valve annulus; a second anchor configured to be positioned proximal to the first anchor and adjacent the mitral valve annulus; and a connecting member attached between the first and second anchors, the first anchor being configured to occupy less than all of the coronary sinus to permit a cardiac lead to be passed by the first anchor. In some examples, the first anchor may include a loop through which the cardiac lead may be passed. In some embodiments, the second anchor is positionable within the coronary sinus and wherein the second anchor is configured to occupy less than all of the coronary sinus to permit the cardiac lead to be passed by the second anchor. In addition, the second anchor includes a loop through which the cardiac lead may be passed. In some examples, the first and second anchors may include a loop through which the cardiac lead may be passed.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic view of a human heart with the atria removed;

FIG. 2 is a schematic view of a human heart comprising a shaping device deployed within a coronary sinus;

FIG. 3 is an enlarged perspective view illustrating one possible embodiment of a shaping device in accordance with the present invention;

FIG. 4 is a perspective view of one embodiment of the invention where an implantable EP system is employed in conjunction with a shaping device;

FIG. 5 is an enlarged view of a shaping device comprising a retention member;

FIG. 6 is a perspective view of a dual function shaping and lead device; and

FIG. 7 is a perspective view of one embodiment of a shaping device comprising a loop member.

### DETAILED DESCRIPTION OF THE INVENTION

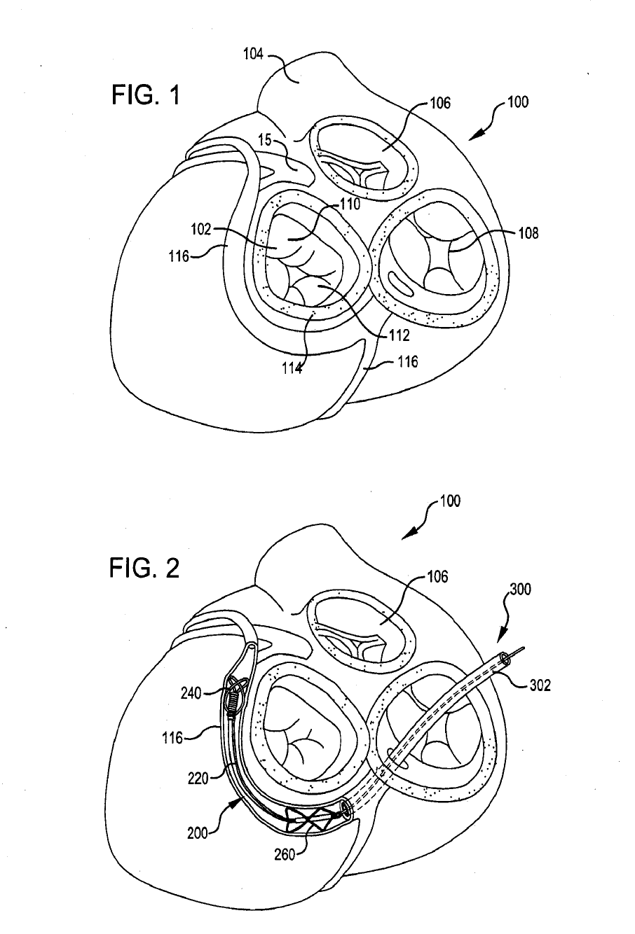

FIG. 1, is a superior view of a heart 100 with the atria removed. It is provided to aid in the understanding of the present invention. As pictured, the heart comprises several valves including mitral valve 102, pulmonary valve 104, aortic valve 106 and tricuspid valve 108.

Turning to mitral valve 102, this valve includes anterior cusp 110, posterior cusp 112 and annulus 114. Annulus 114 encircles cusps 110 and 112 and functions to maintain their respective spacing to ensure complete mitral valve closure during left ventricular contractions of the heart 100. As illustrated, coronary sinus 116 partially encircles mitral valve 102 and is adjacent to mitral valve annulus 114. Coronary sinus 116 is part of the venous system of heart 100 and extends along AV groove between the left atrium and the left ventricle. This places coronary sinus 116 essentially within the same plane as mitral valve annulus 114, making coronary sinus 116 available for placement of shaping device 200 in order to effect mitral valve geometry and to restore proper valve function.

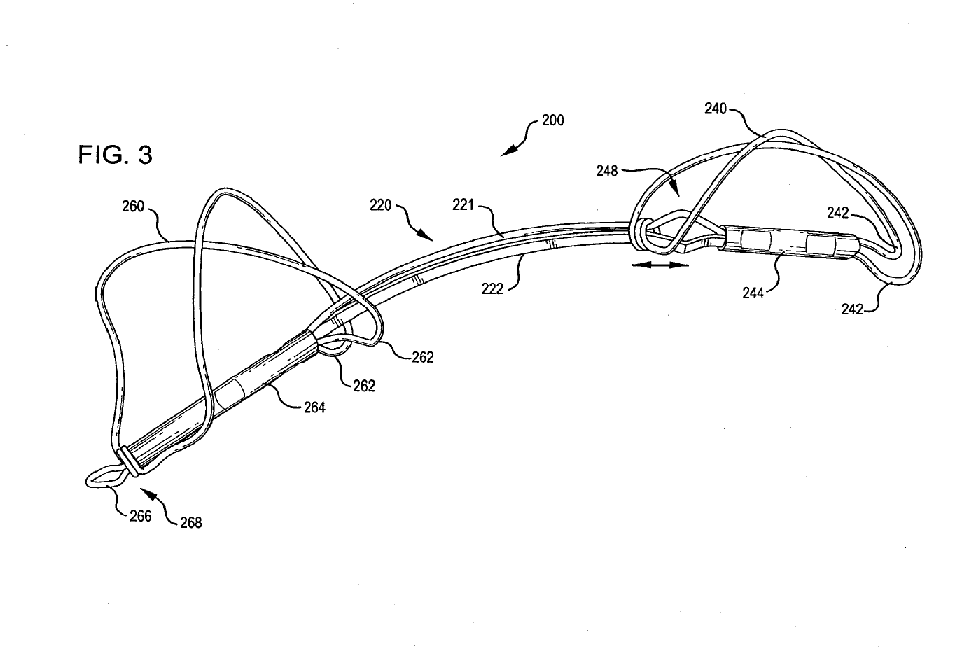

FIG. 2 illustrates one possible embodiment of an implantable shaping device 200, which is deployable in coronary sinus 116 or other body lumen. As illustrated in FIG. 2 (and FIG. 3), device 200 generally comprises elongated connector 220 disposed between distal anchor 240 and proximal anchor 260. Both distal anchor 240 and proximal anchor 260 are shown in their deployed (i.e. expanded) configuration in FIG. 2, securely positioned within the coronary sinus 116. FIG. 2 further depicts, in phantom, a deployment system 300 comprising catheter 302 for delivering and positioning shaping device 200 in the coronary sinus 116.

FIG. 3 is an enlarged perspective view of one embodiment of a shaping device 200. Starting from its most distal to proximal end, shaping device 200 generally comprises: distal strain relief member 242; distal anchor 240; distal crimp tube 244; distal lock feature 248; elongated connector 220 comprising wire connector 221 and ribbon connector 222; proximal strain relief member 262; proximal anchor 260; proximal crimp tube 264; proximal lock feature 268; and proximal lock eyelet 266. Generally, shaping device 200 is configured to be highly fracture resistant by virtue of the one or more stain relief members 242, 262, ribbon connector 222, as well as other features incorporated into the design of shaping device 200.

Generally, shaping device 200 is adapted to be deployed from an opening of a delivery catheter 302 and is anchored within coronary sinus 116 via expansion of distal anchor 240. In one embodiment, distal anchor 240 may be configured to be self-expanding, or alternatively, may be actuated open (via actuation of distal locking mechanism 248). While shaping device 200 is anchored within the coronary sinus 116 after expansion of distal anchor 240 shaping device 200 may be “cinched” as further described in U.S. application Ser. No. 10/742,516, to affect mitral valve geometry sufficient to decrease mitral valve regurgitation. When sufficient alteration of the mitral valve geometry and the leaflets is achieved (as determined by various visual diagnostic techniques including but not limited to fluoroscopy and the like), proximal anchor 260 may be exposed, deployed, expanded, decoupled and catheter 302 withdrawn.

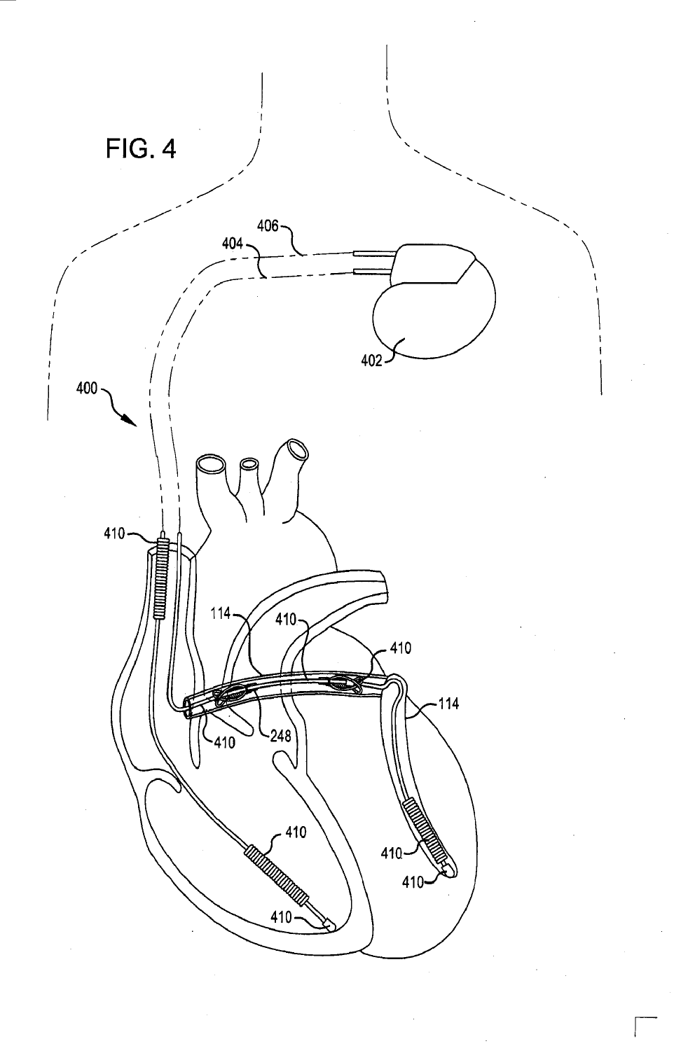

FIG. 4 illustrates an embodiment, wherein shaping device 200 is used in conjunction with an implantable EP system 400. It should be noted that while implantable systems are illustrated herein, external EP systems may be employed without departing from the scope of the present invention. Moreover, the EP system 400 of the present invention may include, but are not limited to: pacemakers, including coronary sinus pacemakers; pulse generators (IPGs); implanted cardioverter-defibrillators (ICDs); pacer-cardioverter-defibrillators (PCDs); and the like. These EP systems may be configured to provide diagnostic and/or therapeutic treatment to a patient.

As shown in FIG. 4, EP system 400 will generally comprises a generator 402, which includes internal circuitry and a power source, such as a battery (not shown), and one or more electrical leads 404 and 406 that couple generator 402 to one or more electrodes 410 disposed on leads 404 and 406. Generator 404 is shown in FIG. 4 as implanted into the pectoral space, just under a patient's collar bone.

Leads 404, 406 are the components of EP system 400 that directly enter the patient's heart, either into the right or left side as needed. Leads 404, 406 comprise one or more electrodes 410 adapted for pacing, sensing and/or defibrillating a patient's heart.

In FIG. 4, EP system 400 is configured as a two lead, implantable cardiac stimulation system for pacing and defibrillating all four chambers of a patient's heart (including for biventricular pacing of the heart), as further described in U.S. Pat. No. 6,760,619, which is herein incorporated by reference in its entirely. As detailed therein, the left lead 404 is provided for sensing, pacing and/or defibrillation of the left side of the heart and is placed within the coronary sinus 116.

In one embodiment, left lead 404 is securely maintained in the coronary sinus 116 by shaping device 200 upon expansion and deployment of said shaping device 200 therein. For example, shaping device 200 can be configured to abut and anchor left lead 404 against an inner wall of the coronary sinus 116, obviating the need for employing lead anchoring techniques. Preferably, in this example, those portions of shaping device 200 that contact left lead 404 should preferably comprise an electrically insulative layer for electrically isolating shaping device 200 from left lead 404. Leads 404 and 406 of EP system 400 may be delivered and implanted using conventionally known techniques.

In some embodiments, leads 404 and 406 may be implanted before the shaping device. For example, shaping device 200 can be implanted into a patient's coronary sinus after a patient has undergone an earlier EP procedure and even after endotheliazation of the leads has occurred. In other embodiments, the EP leads may be implanted after the shaping device. Because of the configuration of shaping device 200, a lead can easily pass through the shaping device because of its open structure and because the shaping device occupies less than all of the interior space of the coronary sinus 116. A further description of this aspect of the invention is described with reference to FIG. 7.

In yet another example, a shaping device 200 and a lead may be delivered simultaneously during a single treatment procedure into a coronary sinus. As will be recognized by those skilled in the art, simultaneous delivery of a shaping device and a lead aids in delivery and positioning of said lead as these devices are not designed for good pushability and therefore can be difficult to deliver and maintain position within a patient's heart, especially during heartbeats.

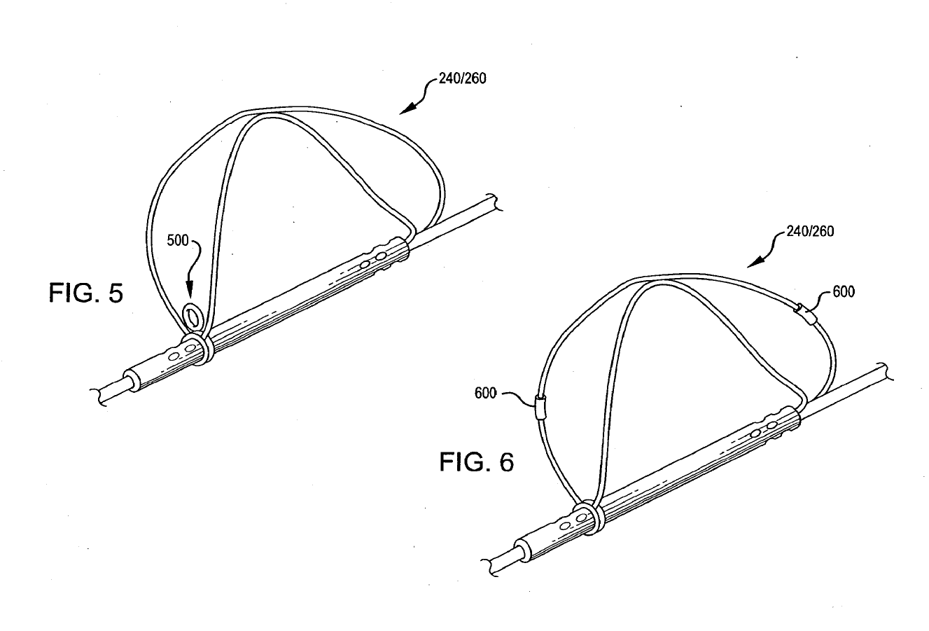

FIG. 5 is an enlarged view of one embodiment of the invention wherein shaping device 200 configured to facilitate simultaneous delivery of a shaping device and lead. As shown, in this embodiment, shaping device 200 comprises a retention member 500. Said retention member 500 may be a hook (not shown), loop (shown), grasper (not shown) or the like for holding a left lead 404 to shaping device 200. While the retention member 500 may be disposed on any portion of the shaping device 200, preferably its placement on device 200 and design ensures that left lead 404 and its electrodes 410 make sufficient electrical contact with tissue so that each electrode's sensing, pacing and defibrillation functions are not compromised. In addition, the retention member 500 is electrically insulated to prevent electrical communication between shaping device 200 and left lead 404.

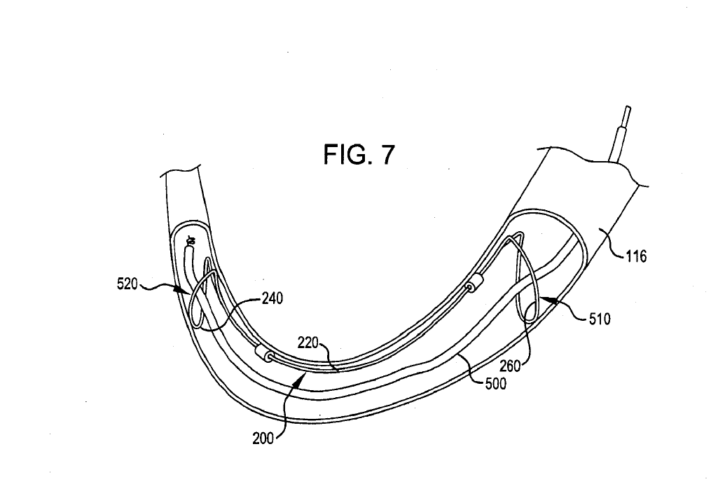

FIG. 6 illustrates yet another embodiment wherein shaping device 200 is configured as a dual cardiac lead and mitral valve treatment device. In this example, shaping device 200 comprises one or more electrodes 600 integrated into shaping device 200. Functionally, the electrodes 600 may be coupled to an EP system 400 and generator 402. Generally, the one or more electrodes 600 may be disposed on any portion of shaping device 200 that makes contact with the vessel wall, including but not limited to: distal anchor 240; proximal anchor 260; both anchors 240 and 260; distal crimp tube 244; proximal crimp tube 264; both crimp tubes 244 and 264; connector 220; or any combination thereof. Alternatively, as components of shaping device 200 may be configured from electrically conductive materials such as nitinol, elastic metals and the like, these components (such as a crimp tube or connector) may be adapted to directly couple to an EP system.

As illustrated in FIG. 6, which shows an embodiment wherein electrodes are disposed on a distal or proximal anchor 240, expansion of said anchor ensures direct and maximal contact between the one or more electrodes 600 and the cardiac tissues to achieve optimal and efficient electrode operation for sensing, pacing or defibrillation of the left side of a patient's heart. Preferably, the electrodes are disposed on a portion of a shaping device that contacts the atrial side of the coronary sinus to ensure proper electrical contact with the left atrium of a patient's heart for pacing, defibrillating and/or sensing.

FIG. 7 illustrates yet another embodiment of the invention, wherein shaping device 200 is configured to permit a cardiac lead and electrode to be passed through the coronary sinus 116. To that end, and as shown in FIG. 7, the anchors 240 and 260 the device 200 occupy only a small portion of and hence less than all of the interior space of the coronary sinus 116. This permits a cardiac lead 500 to be advanced into the coronary sinus 116 for implant in the left side of the heart. More specifically, the anchors 240 and 260 take the form of loops 520 and 510 respectively which are then bent backwards on the device 200 to form hook-shapes for self-deployment. The loops 520 and 510 thus permit the cardiac lead 500 to be passed therethrough for implant in the left heart. This is particularly desirable because many patients suffering from mitral regurgitation may also be candidates for left heart cardiac rhythm management therapy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for treating a condition of the heart and which is configured to be deployed in a coronary sinus, said device comprising: expandable first and second anchors interconnected by a connecting member disposed between the first and second anchors; and a retention member adapted for holding a cardiac lead in a coronary sinus.

2. The device of claim 1, wherein the retention member is a loop, hook, grasper or the like.

3. The device of claim 2 wherein the cardiac lead is an IPG, ICD, PCD or pacemaker lead.

4. A device for treating a condition of a patient's heart and which is configured to be deployed in a coronary sinus, said device comprising:
an expandable distal anchor; an expandable proximal anchor; a connecting member disposed between the distal and proximal anchors; one or more electrodes; and a lead wire which operationally couples the one or more electrodes to an EP system.

5. The device of claim 4 wherein the distal and proximal anchors further comprise a distal and proximal crimp tube respectively.

6. The device of claim 4 wherein the one or more electrodes are located on the distal anchor, the proximal anchor or both.

7. The device of claim 5 wherein the one or more electrodes are located on a connecting member, a crimp tube or both.

8. The device of claim 4 wherein one or more of the crimp tubes, connector or both are adapted to couple to the EP system.

9. The device of claim 4 wherein the EP system is an IPG, ICD, PCD or pacemaker system.
